(19) United States
(12) Patent Application Publication
(10) Pub. No.: US 2023/0047610 A1
(43) Pub. Date: Feb. 16, 2023

(54) DEVICES AND METHODS FOR SELECTING STENTS

(71) Applicant: DP Holding (U.K) Limited, Grantham (GB)

(72) Inventors: Peter BALMFORTH, Oslo (NO); Darren SPENCER, Oxford (GB); Paul SOBOTKA, Philadelphia, PA (US); Rodney BRENNEMAN, San Diego, CA (US); Nathan SOBOTKA, Minneapolis, MN (US)

(21) Appl. No.: 17/797,586

(22) PCT Filed: Feb. 4, 2021
(86) PCT No.: PCT/US2021/016563

§ 371 Date: Aug. 4, 2022

Related U.S. Application Data
(60) Provisional application No. 62/969,946, filed on Feb. 4, 2020.

(51) Int. Cl. A61B 5/02; A61B 5/00; A61F 2/82

(52) U.S. Cl. CPC A61B 5/02007; A61B 5/0053; A61B 5/6853; A61F 2/82; A61B 2034/108

(57) ABSTRACT

A catheter-based device for determining the radial expansion force required to displace an occlusion in a vessel located in a subject. The device comprises an elongate body defining proximal and distal termini. The body comprises a sheath that encloses a hollow lumen within, which extends along substantially the full length of the body. The proximal terminal region of the body comprises: a user-interfacing hub, the hub comprising a handle for maneuvering the body and configured for handling by an operator; a control interface for controlling the device; and a sensor configured to measure one or more parameters relevant to a force applied to the vessel by the device. The distal terminal region of the body comprises an expandable member movable between a retracted position, in which the expandable member is within the hollow lumen, and a deployed position, in which the expandable member is disposed beyond the distal terminus, and controllable via the control interface to expand radially. The expansion of the expandable member is correlated to a defined radial expansion force value.

### TECHNICAL FIELD

The present disclosure relates to devices and methods for selecting stents for vessels, particularly devices and methods for determining required radial forces to select an appropriate stent for a target vessel.

### BACKGROUND

The technique of percutaneous transluminal coronary angioplasty has been extensively used since the 1980s to restore blood flow in blocked arteries. It is a relatively common percutaneous technique that is performed on a daily basis around the world. While angioplasty is typically used in the coronary arteries to restore flow, the techniques have also been applied to peripheral arteries. In 1960, Charles Dotter developed the first balloon-based catheter to dilate the narrowed arteries of the leg to allow passage of ever-increasing diameters of catheters. In 1973, the first balloon catheter designed for the iliac artery was developed by physicians from the University Hospital of Zurich.

The typical coronary angioplasty is performed under local anaesthetic with a thin tube inserted into the arteries of the heart with a balloon mounted onto the tip and shaft of the catheter. The balloon is inflated via the use of a manometer to a specific pressure. Once the artery has been sufficiently stretched, a stent is inserted to keep the artery open and to preserve blood flow. Stenting is common in modern angioplasty.

Whilst the field of coronary stenting has been developed over several decades and balloon-based catheters have been used in peripheral arteries, the field of venous and peripheral vascular stenting is still in its infancy. Peripheral venous vasculature presents a range of anatomical challenges that were previously unseen in coronary arterial stenting. Important consideration factors are the large lumen diameters, long stent lengths, flexible venous walls that are vulnerable to compression by external structures, and the highly mobile locations of the body in which the vessels are found. All these factors require precise positioning and stability of the stent, as well as radial force application by the stent to overcome the lesion. However, stents that impede natural movement and the underlying anatomy should be avoided. These factors necessitate a unique and personalized approach to stenting and angioplasty strategies to ensure not only excellent primary and secondary patency rates, but also without risk of making the individual worse through stent failure. There are now multiple manufacturers of arterial and venous stents, each with unique design features and builds in order to provide “their solution” to the problem. However, the procedure for selecting the correct stent to overcome the occlusion/compression is essentially guesswork.

The difficulty of correctly choosing the right stent for deployment in peripheral vasculature was previously complicated by the presence of numerous devices designed for use in coronary artery stenting and the absence of specifically designed peripheral venous stents. The different requirements for venous and peripheral stenting when compared to coronary artery stenting mean that the available equipment did not address the unique features and requirements of stent placement, radial force and flexibility needed for success in peripheral venous applications. In a great many instances, clinicians resort to using stents designed originally for use in the arterial system, repurposing them for venous use. This has resulted in poor patient outcomes as in certain cases the implanted devices are simply not fit for purpose. In recent years, manufacturers who have developed dedicated venous stents have provided their own solution to overcome the venous challenges. However, to date, no one stent manufacturer has developed a single ideal stent. Stenting into the common femoral vein requires a woven, braided stent to prevent stent fracture and flexibility, where a laser cut nitinol stent could potentially fracture. Conversely a venous compression such as a NIVL or May-Thurner compression requires a large degree of radial force to overcome. Radial force is typically superior in laser cut nitinol stent compared to that of the woven braided stent. Additionally, the overall goal to restore flow through an occluded venous segment, necessitates the aim to achieve a stent that is as circular in shape as possible, to give the best inflow/outflow and prevent in-stent restenosis. This in itself requires high degrees of radial force, which may impede free movement of the individual, inflict long-standing pain through oversizing stents and/or cause premature stent failure due to increased torsional forces on the stent. So there is a delicate balance that needs to be found, in order to appropriately choose the right stent for the right anatomy and to overcome the specific occlusion.

In another example, modern balloons used in balloon-based catheters are manufactured from multiple different types of materials to meet the needs and requirements of the end product and its intended purpose. These include, but are not limited to: polyethlene terephthalate (PET); polyolefin copolymer (POC); nylon; polyether block amide (PEBA OR PEBAX®); silicone; and other compound polyurethanes. This is a change from initial balloon-based catheters, which were initially made of flexible polyvinyl chloride (PVC), and then in the second generation cross-linked polyethylene (PEX).

So, complexity is first introduced by the sheer number of different raw materials. There are also multiple methods in which to build or construct the balloons, including but not limited to: extrusion; moulding; and dip casting. Different balloon properties are conferred depending on upon which process is used for manufacturing. The balloons can also be manufactured in multiple lengths, diameters, shapes, profiles, and coatings to achieve the desired properties.

Regardless of intended use, manufacturers have grouped the various types of balloons into 3 broad categories based on the intended use applications: compliant balloons; non-compliant balloons; and semi-compliant balloons. In compliant balloons, the diameter of the balloon increases proportionally to the increase in inflation force. The size of a compliant balloon may grow beyond the ceiling of clinical safety. In non-compliant balloons, the diameter of the balloon is highly restricted, so that only small changes in diameter are possible. Semi-compliant balloons have a wide working pressure range with controlled flexibility in balloon sizing.

Typically a balloon of a single manufacturer has specific characteristics, but may differ significantly from those of other manufacturers. As an example, there are significant and expected differences in compliance between the three specific types of balloons i.e. compliant, non-compliant and semi-compliant. Over a range of increasing pressures, the diameter of a non-compliant balloon is relatively constant but the diameters of semi-compliant and compliant balloons are much more variable.

This is further complicated when considering balloons of the same size but of different manufacturers, as nominal pressure and burst pressure for each balloon vary quite considerably. As complexity in balloons is now high, there may also be non-negligible differences in diameter at the nominal pressure between balloons of the same types. This is because manufacturing complex balloons in a repeatable manner is much more difficult.

Moreover, because arteries are resilient vessels that can withstand the relatively high forces placed on them by balloons and stents without collapse or disintegration, there remain relatively basic methods of inflation and measurement of balloon size by translating balloon pressure to lumen diameter. However this often leads to vessel and stent overexpansion in order to overcome recoil. Overexpansion may result in increased endothelial damage and increased rates of in-stent restenosis, especially in peripheral vasculature and the venous system. Accordingly, there is a desire to improve the techniques used in venous and peripheral angioplasty so that safety of the patient is ensured and maintained.

It is an aim of the present invention to address one or more of the disadvantages associated with the prior art.

### SUMMARY OF THE INVENTION

According to an aspect of the present invention there is provided a catheter-based device for determining the radial expansion force required to displace an occlusion in a vessel located in a subject. The device comprises an elongate body defining a proximal and a distal termini, the body comprising a sheath that encloses a hollow lumen within, which extends along substantially the full length of the body. The proximal terminal region comprises: a user-interfacing hub, the hub comprising a handle for maneuvering the body and configured for handling by an operator; a control interface for controlling the device; and a sensor configured to measure one or more parameters relevant to a force applied to the vessel by the device. The distal terminal region comprises: an expandable member movable between a retracted position, in which the expandable member is within the hollow lumen, and a deployed position, in which the expandable member is disposed beyond the distal terminus, and controllable via the control interface to expand radially. The expansion of the expandable member is correlated to a defined radial expansion force value.

According to another aspect of the invention, there is provided a method for determining the radial expansion force required to displace an occlusion in a vessel located in a subject. The method comprises: providing a catheter-based device having an expandable member expandable to apply force to the occlusion; disposing the expandable member within the vessel in the region of the occlusion; expanding the expandable member to achieve a target profile within the lumen, wherein the expansion of the expandable member is correlated to a defined radial expansion force value; and determining the radial expansion force value applied by the expandable member to the lumen to achieve the target profile based on the correlation.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 shows a vessel with a compression that is being treated by a stent;

FIGS. 2A to 2C show (A) simultaneous arterial and venous contrast injection in a therapy resistant hypertensive patient, with no signs or symptoms of leg swelling (LAO orientation). (B) and (C) demonstrate impeded contrast flow in the vein via direct overriding arterial compression taken from both AP and LAO angles respectively; white arrows show the location of the venous obstruction;

FIG. 3 shows a system including a device for determining radial force according to an embodiment of the invention;

FIGS. 4A to 4E show the use of the device of FIG. 3 within a target vessel according to an embodiment of the invention;

FIG. 5 shows a distal end of a catheter with an inflated balloon according to an embodiment of the invention;

FIG. 6 shows a distal end of a catheter with an inflated balloon according to another embodiment of the invention;

FIG. 7 shows a distal end of a catheter with an inflated balloon according to another embodiment of the invention;

FIG. 8 shows a distal end of a catheter with an inflated balloon according to another embodiment of the invention;

FIG. 9 shows a distal end of a catheter with an inflated balloon according to another embodiment of the invention;

FIG. 10 shows a distal end of a catheter with a deflated balloon according to an embodiment of the invention;

FIG. 11 shows a distal end of a catheter with a deflated balloon according to another embodiment of the invention;

FIG. 12 shows a distal end of a catheter with a deflated balloon according to another embodiment of the invention;

FIGS. 13A to 13D show different mechanisms for positioning a balloon relative to a compression of a target vessel;

FIG. 14 illustrates a flow chart governing the use of the device in determining radial or local force according to an embodiment of the invention;

FIG. 15 shows a distal end and a proximal end of a catheter with a basket according to an embodiment of the invention;

FIG. 16 shows a distal end and a proximal end of a catheter with a basket according to another embodiment of the invention; and

FIG. 17 shows a distal end and a proximal end of a catheter with a basket according to another embodiment of the invention.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

### Definitions

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “kink resistance” refers to a stent's ability to withstand mechanical bending loads from the surroundings depending upon the position in the body. Usually, this is based upon the smallest radius of curvature a stent can withstand without the formation of a kink. In areas of high tortuosity within the body it is necessary for a stent to have increased kink resistance to prevent a reduction in lumen patency or even total occlusion.

The term “crush resistance” refers to the ability of a stent experiencing external, focal or distributed loads to resist collapse. These loads ultimately lead to stent deformation and even full or partial occlusion which can result in adverse clinical consequences. Crush resistance of an endovascular device may be measured using the parallel plate method to determine the effective load required to reduce the luminal diameter by 50% as described in ISO 25539-2.

The term ‘obstruction’ or ‘occlusion’ refers to any occurrence whereby the diameter (or ‘caliber’) of a vessel is reduced when compared to a normal, i.e. non-occluded, state. Venous obstruction can occur through the narrowing (stenosis) of a vein, through blockage or through externally applied pressure causing a localised compression of the vein. The term also includes venous occlusion, whereby the vein's lumen is partially or totally obstructed to the flow of blood. Occlusion may result from thrombosis (e.g. deep vein thrombosis (DVT)) or may be due to tumour incursion. The term also includes ‘venous compression’, which refers to the external compression of the vein. The source of external compression may be caused by an adjacently located artery compressing the vein against another fixed anatomical structure, which can include the bony or ligamentous structures found in the pelvis, the spine itself, or overlapping arterial branches. External compression may also arise from tumours, growths, glands, developing foetuses and/or other developing mass that may occur within the pelvic space.

The term ‘venous return’ is defined by the volume of blood returning to the heart via the venous system, and is driven by the pressure gradient between the mean systemic pressure in the peripheral venous system and the mean right atrial pressure of the heart. This venous return determines the degree of stretch of heart muscle during filling, preload and is a major determinant of cardiac stroke volume.

The term ‘May-Thurner syndrome’ (MTS) also known as iliac venous compression syndrome (which includes Cockett's syndrome) is a form of ilio-caval venous compression wherein the left common iliac vein is compressed between the overlying right common iliac artery anteriorly and the lumbosacral spine posteriorly (fifth lumbar vertebra). Compression of the iliac vein may cause a myriad of adverse effects, including, but not limited to discomfort, swelling and pain. Other less common variations of May-Thurner syndrome have been described such as compression of the right common iliac vein by the right common iliac artery; this is known as Cockett's syndrome. More recently, the definition of May-Thurner syndrome has been expanded to include an array of compression disorders associated with discomfort, leg swelling and pain, without the manifestation of a thrombus. Collectively, this has been termed non-thrombotic iliac vein lesions (NIVL).

The term ‘intraluminal thickening’ (also referred to as venous spurs or intraluminal spurs) is related to this external compression of the left common iliac vein by the right common iliac artery against the fifth lumbar vertebra. Venous spurs arise due to the chronic pulsation of the right common iliac artery. This ultimately results in an obstruction to venous outflow. Venous spurs are internal venous obstructions consequent to chronic external compression of veins by adjacent structures.

The term ‘Deep Vein Thrombosis’ (DVT) refers to the formation of blood clots or thrombus within the venous segment, and in itself is not life threatening. However, it may result in life threatening conditions (such as pulmonary embolism) if the thrombus were to be dislodged and embolize to the lungs. Additionally, DVT may lead to loss of venous valvular integrity, lifelong venous incompetence and deep venous syndrome which includes rest and exercise pain, leg swelling and recurrent risk of DVT and emboli. The following is a non-limiting list of factors that reflect a higher risk of developing DVT including prolonged inactivity, smoking, being dehydrated, being over 60, undergoing cancer treatment and having inflammatory conditions. Anticoagulation which prevents further coagulation but does not act directly on existing clots, is the standard treatment for deep vein thrombosis. Other potentially adjunct, therapies/treatments may include compression stocking, selective movement and/or stretching, inferior vena cava filters, thrombolysis and thrombectomy.

The term “nominal pressure” is the balloon inflation pressure at which the balloon reaches its stated size without external influence.

The term “rate burst pressure” is the balloon inflation pressure at or below which 99.9% of balloons of that type will not burst.

The term “working range” is the range of balloon inflation pressures between the nominal and rate burst pressures.

The term “compliant” refers to balloons whose diameter increases proportionally to the increase in pressure within the balloon.

The term “non-compliant” refers to balloons that expand to an intended size as internal pressure increases. Once the balloon reaches its intended size, its size does not change further. These balloons are generally used to transmit force on a lumen wall or displace an extrinsic compression.

The term “semi-compliant” refers to balloons that expand to a range of size as the internal pressure increases.

### DESCRIPTION OF EMBODIMENTS

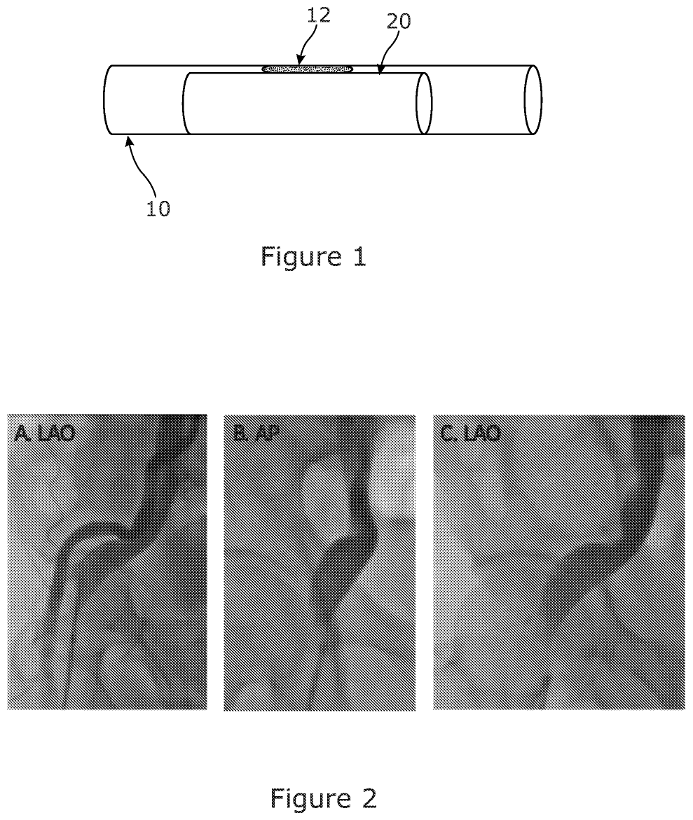

FIG. 1 shows a schematic representation of a blood vessel 10 incorporating a stent 20. The blood vessel 10 may be an artery or a vein, or even a non-vascular duct. The vessel 10 has an occlusion 12. Although referred to as an occlusion here, the occlusion may alternatively be a region of stenosis, a compression of the vessel, a reduced calibre caused by an external force pressing on the vessel 10, or anything else that causes a closing or constriction of the lumen of the vessel 10 that is detrimental to its flow characteristics. To restore the lumen of the vessel 10 to its conventional diameter and shape, a stent 20 is positioned within the lumen of the vessel 10 and in direct contact with the tissue forming the vessel 10. The stent 20 acts to reduce the impact of the occlusion 12 on the flow of blood through the vessel 10. The stent 20 expands the vessel 10 to an aspect ratio of close to or exactly 1.0 at a diameter that is similar to the surrounding, healthy, undilated tissue of the vessel 10. This undilated tissue is typically found downstream of the occlusion where no congestion is present in the vessel 10. An aspect ratio of ˜1.0 ensures continuity of flow through the vessel 10 without a restriction in the velocity of the flow of blood. An aspect ratio of ˜1.0 also ensures that turbulence is avoided in the flow. An aspect ratio of substantially 1.0 may be considered to be an aspect ratio of between 0.9 and 1.1, or more preferably between 0.95 and 1.05.

In an example of a constriction of a vessel, an individual may have no apparent signs or symptoms of leg swelling but, nevertheless, an obstruction or compression of the veins in the ilio-caval region may be suspected. Normal anatomy in this region sees the vein assume an upward sigmoidal curve from the femoral vein to the inferior-vena cava. In FIG. 2A-C an example of arterial compression of an adjacent underlying vein is observed using contrast fluoroscopy. It would be apparent to the skilled person that a solution is required that allows for the restoration of luminal patency and normal blood flow. The skilled person may understand that relieving the obstruction in this region by implanting a stent with low flexibility and high crush resistance would profoundly alter the local anatomy and may not be in the best interests of the body and in the longer term could induce restenosis and intimal hyperplasia resulting in stent failure and more severe venous occlusion. The skilled person may therefore understand that a highly flexible stent with one or more reinforced regions positioned only at the specific points where the compressions are observed (see white arrows in FIG. 2C) would be the requirement for the stent. The reinforced regions may be provided either as integrated within the stent or as individually positionable reinforcing stent elements.

However, the challenge lies in deciphering, from these images alone, the characteristic values that a stent positioned within the vessel should apply to the vessel, such as the outwardly radial force or crush resistance. An under-performing stent will have negligible effect, while an overzealous stent that applies too high forces on the vessel will be detrimental to the health of the patient. It is currently difficult for physicians to assess the potential success for a given stent to adequately restore luminal diameter. It is currently only after placement of the chosen stent that a physician may realize that force applied by the stent is unsuitable for the vessel. A stent applying insufficient force to resist the compression will not correct the vessel's obstruction adequately. A stent applying a too high force may deform the vessel into an undesirable shape or may cause damage to the vessel itself, causing collapse or further complications.

Accordingly, the inventors have devised means for determining a target force to be applied by a stent deployed in the target vessel 10 at the site of the occlusion. In determining a target force, a medical practitioner is able to select a stent for placement within the lumen of the target vessel 10 in order to restore normal or near-normal blood flow past the occlusion 12. While existing systems rely on assessing imagery alone to effectively guess which stent to choose, the approach described herein provides data from several sources to enable a more precise stent choice to be made.

In general, the systems devised by the inventors comprise a catheter or catheter-based device, which may be referred to as a force catheter, configured to be passed along the target vessel. An elongate body of the catheter device comprises a proximal terminus region comprising a user-interface hub and a control interface for controlling the progress and operation of the catheter. The user-interface hub and/or control interface may comprise a handle of the catheter for handling the device and maneuvering the device by an operator. The control interface may comprise one or more controls for enacting actions to performed using the catheter device. At a distal terminus region, an expandable member, also referred to as a vessel expander, is mounted to a main shaft of the catheter device. The expandable member is configured to be deployed from a hollow lumen of the elongate body to extend beyond the distal terminus of the elongate body. The expandable member is configured to expand in order to move the target vessel to a target profile, i.e. to a target aspect ratio, generally an aspect ratio of approximately unity (i.e. 1), and to a target diameter. The expandable member expands within the target vessel to expand the lumen of the vessel and to restore patency of the target vessel. In expanding the target vessel, the expandable member applies a force to the interior of the lumen in the region of an occlusion. The force applied by the expandable member on the target vessel to achieve the target profile may be measured either directly or indirectly based on the operation of the expandable member using a measurement device associated with the expandable member. The systems may also include one or more imaging systems to enable imaging and therefore guidance of the catheter within the target vessel. The force applied by the expandable member, namely the radial expansion force, is correlated to the expansion of the expandable member and can be determined accordingly.

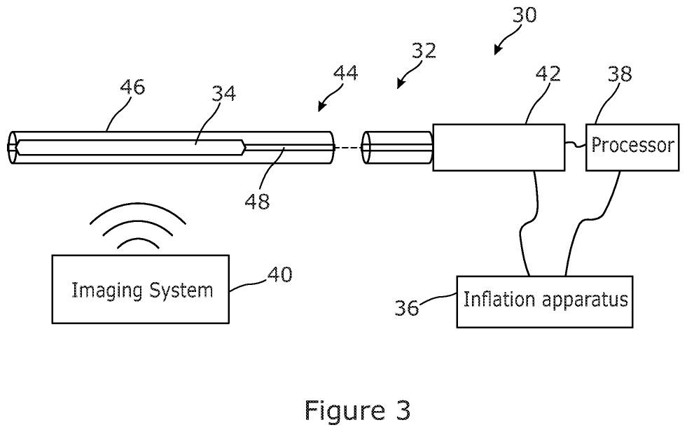

An example system 30 is shown in FIG. 3. The system 30 of FIG. 3 has a catheter 32 including an expandable member 34 in the form of an inflatable balloon, inflation apparatus 36 for inflating and deflating the balloon, a processor 38 connected to the catheter 32 and the inflation apparatus 36, and an imaging system 40.

The imaging system 40 may be any suitable system for use in imaging the target vessel 10 and/or parts of the catheter device 32. The imaging system 40 may include an Intravascular Ultrasound (IVUS), an Optical Coherence Tomography (OCT), a contrast fluoroscopy systems, or other imaging modality or a combination of these. IVUS and OCT are preferable as they are typically used to determine vessel size and lumen size accurately.

It should be noted that, as indicated in FIG. 3, the imaging system 40 is separate from the catheter device 32 itself. The imaging system 40 is used to visualize the catheter 32 as it progresses along the target vessel 10 and to identify when the catheter 32 is correctly positioned. The imaging system 40 may also be used for preparatory investigations prior to insertion of the catheter 32 into the patient's body, and even prior to selection of a balloon size for use in dilating the target vessel 10.

In some embodiments the imaging system 40, or part of the imaging system 40, may be incorporated into the catheter device 32 itself. In these embodiments, a central lumen of the catheter 32 may be dimensioned to accommodate an IVUS catheter such that IVUS can be used at the same time as the balloon is being positioned and inflated. There may be one or more slotted windows along the shaft of the delivery catheter 32 that allow for visualization with IVUS if available for precise positioning of the balloon.

The processor 38 may receive data output from the inflation apparatus 36, the imaging system 40, and/or one or more sensors in the catheter 32. The processor 38 may analyze the received data to determine a radial force that a stent 20 should apply to the occluded target vessel 10 to overcome the occlusion. The processor 38 may perform one or more further actions, as will be discussed below. In some examples, the processor 38 instead may be configured to convert the output data it receives into charts for interpretation by a medical practitioner instead of or in addition to the determination of radial force. The charts generated may be displayed on a display device.

Turning now to the catheter device 32, the catheter device has a handle 42 and a catheter body 44. The handle 42 is positioned at a proximal end of the device 32. The handle 42 is attached to the elongate catheter body 44 that extends to a distal end of the device 32. The handle 42 is utilized by the user of the device, typically a medical practitioner, to control and manoeuvre the catheter body 44. The catheter body 44 connects to the handle 42 at its proximal end. The catheter body 44 is configured to be delivered along the lumen of the target vessel 10. The distal end of the catheter body 44, forming the distal end of the device 32, is a free end. In some embodiments, the catheter body 44 may be passed over a guide wire (not shown in FIG. 3). The use of a guidewire is discussed in relation to later embodiments.

Catheter bodies, such as the catheter body 44 of FIG. 3, are suitably constructed in a variety of sizes typically ranging from 0.6 mm up to 3.33 mm in diameter (corresponds to French sizes 2 to 10). Guidewires for use with catheters of the invention are typically in the size range of 0.05 mm to about 1 mm (about 0.002 inches to about 0.05 inches). The catheter body is suitably manufactured from plastics or polymeric biocompatible materials known in the technical field, for example, PTFE. In one embodiment of the invention (not shown), the device catheter body may be manufactured from a flexible material so as to enable the device to follow the natural curvature of the lumen of the vessel through which it is travelling.

The catheter body 44 in FIG. 3 comprises an introducer sheath 46. The introducer sheath 46 has a central lumen, within which a shaft 48, such as a hypotube, is provided. The introducer sheath 46 and shaft 48 are capable of being advanced together along the target vessel 10. As required, the sheath 48 may be withdrawn to expose the distal end of the shaft 46 carrying an expandable member 34. The shaft 46 may alternatively be capable of being advanced beyond the end of the sheath 48. In either deployment, the relative movement is enacted and controlled remotely, either using controls at the handle 42 or otherwise.

An expandable member 34 is provided at the distal end of the shaft 48. The shaft 48 and expandable member 34 may together be advanced over a guidewire deployed along the target vessel 10. In the case of FIG. 3, the expandable member 34 is a balloon. When the shaft 48 and expandable member 34 are within the sheath 46, the expander 34 is in an unexpanded state to allow passage along the target vessel 10. The balloon is in the unexpanded state when it is deflated and folded to fit within the lumen of the sheath.

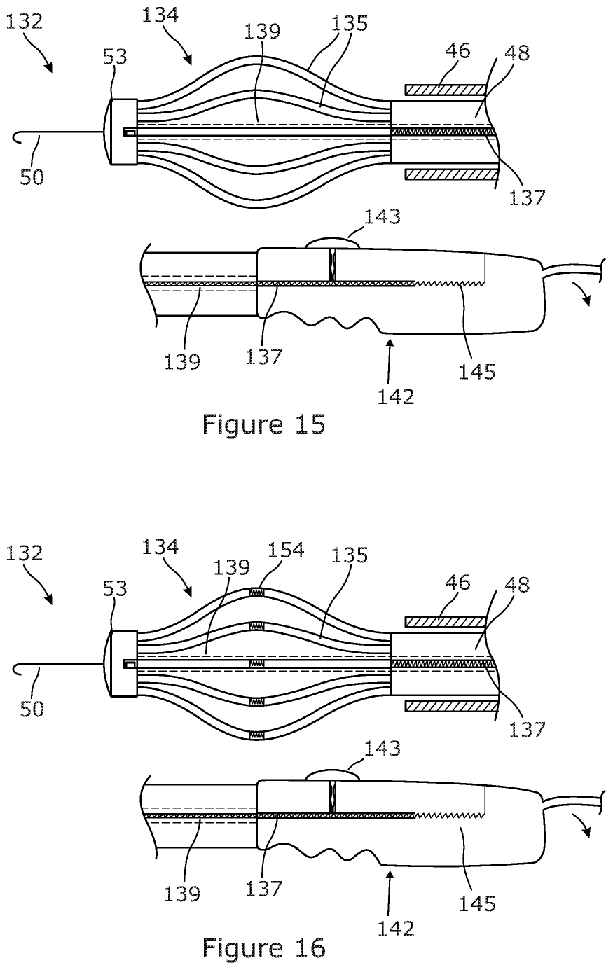

In some embodiments, as will be described later other expandable members may be used instead of the balloon. Expandable members that may be used in this device include the basket arrangement of FIGS. 15 to 17. Other expandable members such as coils, tethered expandable stents or helical basket arrangements that are mountable to the shaft and where the force applied to the vessel by the expander can be quantified may be used in conjunction with this device and instead of the balloon.

For now, returning to the embodiment of FIG. 3, in which the expandable member 34 comprises the balloon, the balloon is capable of being inflated and deflated using the inflation apparatus 36 connected to the device 32. The inflation apparatus 36, generally a manometer and/or another inflation device and pressure gauge, inflates the balloon by passing a pressurised solution along an internal lumen that extends along the shaft 48 to permit fluid communication with the inside of the balloon. The pressurised solution is typically a mixture of saline solution and a contrast agent. In some embodiments, a gas may be used to inflate the balloon. The inflation apparatus 36 is configured to inflate the balloon while measuring the pressure within the balloon. To deflate the balloon, the inflation apparatus 36 allows venting of the pressurised solution from the balloon via the lumen in the shaft 48.

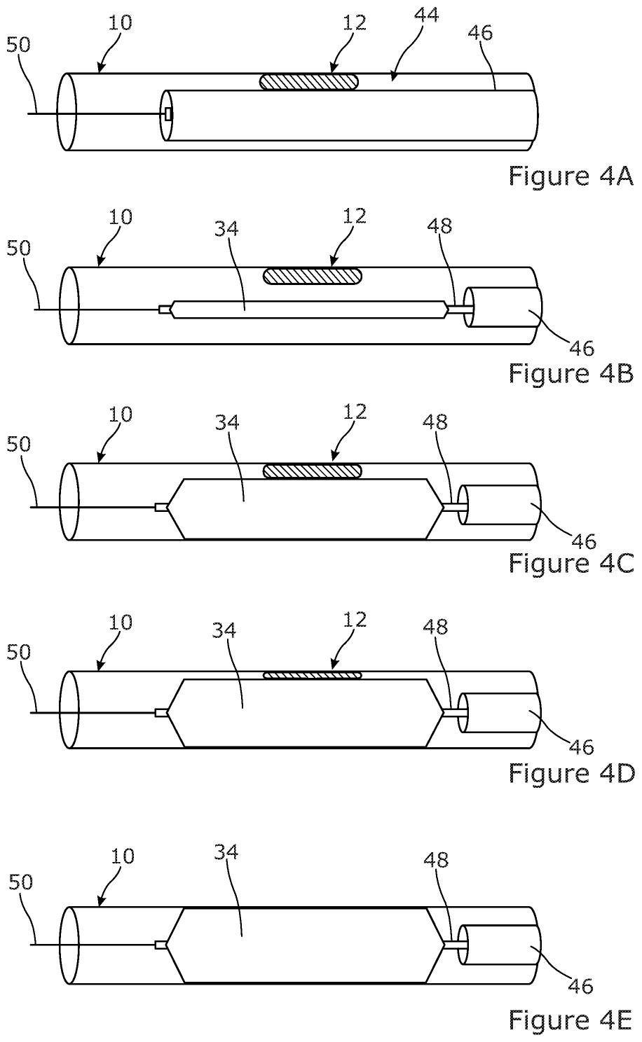

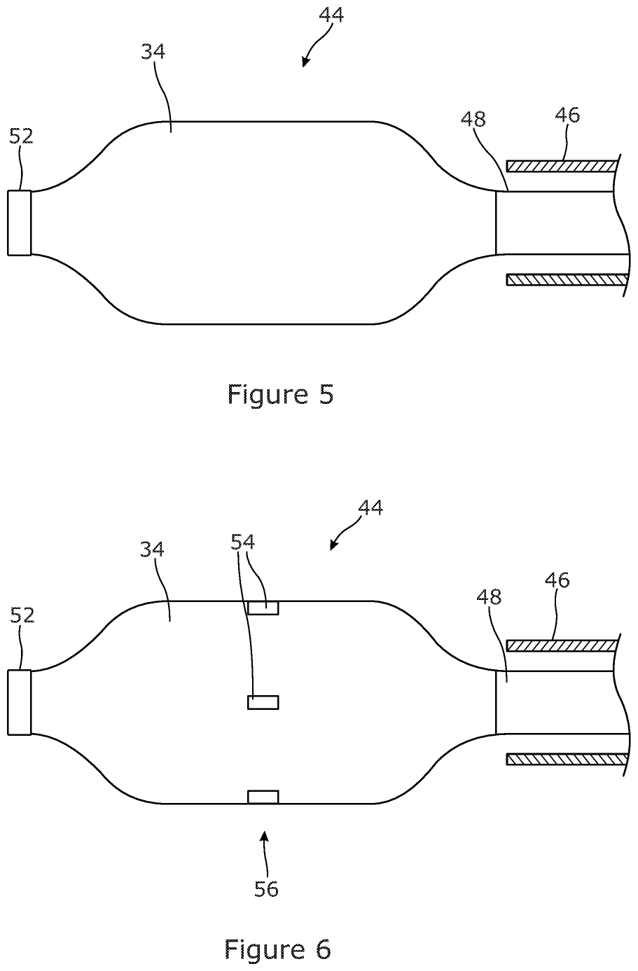

FIGS. 4A to 4E illustrate a positioning and inflation of the balloon 34 within the target vessel 10. FIG. 5 provides another representation of the inflated balloon 34 as part of the catheter 32.

It should be noted that FIGS. 4A to 4E are schematic depictions only and that the interaction of the balloon 34 with the obstruction may be different in practice. In particular, although the obstruction is shown as getting smaller in FIGS. 4D and 4E, this is meant to only be representative of an opening of the lumen to restore patency of the vessel. In practice, the balloon 34 is likely to displace the obstruction and vessel wall to restore the internal diameter.

Initially, the catheter body 44 is advanced along the target vessel 10 until it reaches the occlusion 12 as shown in FIG. 4A. Once the occlusion 12 has been reached, the sheath 46 is drawn back to expose and deploy the expandable structure of the expandable member, in this case the balloon 34, as shown in FIG. 4B. The balloon 34 is then inflated. The inflation of the balloon 34 is performed in stages, as will be discussed in more detail later. The balloon 34 is inflated in stages until the balloon 34 has restored the target profile of the target vessel 10. When the vessel 10 has reached the target profile, the balloon 34 will also have the target profile. In FIGS. 4C and 4D, the target profile has not yet been reached—it can be seen that the balloon 34 is not the same diameter as the healthy tissue either side of the occlusion 12. In FIG. 4E, the target profile has been reached. At this point, the balloon 34 has been inflated to a point at which the target profile of the vessel 10, i.e. the aspect ratio of ˜1.0 and the target diameter of the surrounding tissue, has been achieved. Generally, this involves the balloon 34 moving the occlusion 12 in order to re-open the vessel, and thus restore optimal flow. In displacing the occlusion 12, the balloon 34 is effectively performing the role that the stent 12 will later perform on a more permanent basis. Once the target profile has been reached the force applied by the balloon 34 when the target profile has been reached is determined.

The force applied by the balloon 34 is determined, in this embodiment, by measuring the hydrostatic pressure within the balloon 34 and correlating this pressure with an applied force. The correlation may be performed by the processor 38 and may be based on log tables or charts generated by experiments. In other embodiments, other mechanisms for determining the force may be used, such as a measurement from a direct or indirect force sensor provided on the expandable member. Based on the force readout necessary to displace the occlusion 12 and restore vessel patency, a medical practitioner can select an appropriate stent for implanting within the vessel to apply a similar force. Physical properties of venous stents are known, for example see Dabir et al. (Cadiovasc Intervent Radiol (2018) June; 41(6): 942-950).

In certain instances, the force applied by the expandable member to the target vessel may be the force required to displace an extrinsic compression and/or kink in a primary stent and/or another obstruction.

The balloon 34 has specific properties that permit it to be used as an expandable member within the context of this application. In other words, the balloon is specifically designed so that the pressure therein is correlatable with the force it imparts upon the lumen of the target vessel. Properties of angioplasty balloons and testing methods associated therewith are described in ISO 25539.

Particularly, in embodiments of the invention, the balloon 34 is a non-compliant balloon. Non-compliant balloons inflate to a predetermined size and shape. Once the predetermined size and shape are reached further expansion of the balloon with increasing pressure is negligible until the burst pressure is reached. Because of its non-compliance, the balloon 34 is capable of applying a force to the lumen wall in order to expand the target vessel in which it is deployed. As the balloon 34 is selected to have a diameter substantially equivalent to the diameter of the unoccluded target vessel 10 and the diameter of non-compliant balloons once fully inflated remains substantially the same at pressures below the burst pressure, the balloon 34 having will not dilate the target vessel but will apply a force to restore the target vessel to the target profile and aspect ratio.

To enable general use of the balloon 34, there is a repeatable correlation of the balloon's pressure with the force it applies to overcome the occlusion 12. This is achieved by careful design of the balloon combined with the inflation apparatus enabling accurate determination and control of the pressure within the balloon 34. Careful design of the balloon 34 is achieved by adhering to strict manufacturing tolerances to ensure each balloon has substantially similar inflation and deflation characteristics. The high standards applied in these balloons means that inflation of each balloon is highly repeatable and that the pressure within each balloon can be correlated to the radial expansion force applied to the target vessel 10 upon deployment.

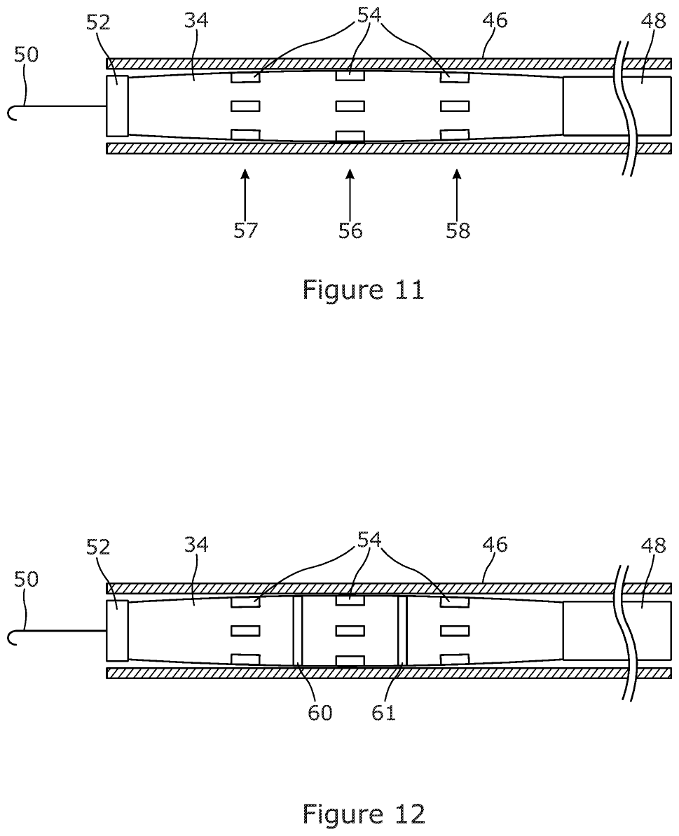

In addition, it can be seen in FIG. 5 that the catheter body 44 has a rounded tip or nose 52 at its distal end. The rounded nose 52 prevents trauma being caused to the vessel 10 should it come into contact with the wall of the vessel 10. To be clear, FIG. 5 also illustrates an inflated balloon 34, the shaft 48 to which the balloon 34 is mounted and the introducer sheath 46. The balloon 34 is depicted in the deployed and inflated state in FIG. 5. The vessel and occlusion are not shown in FIGS. 5 to 12.

In addition to sensing the force, it is also important to understand how the vessel 10 and balloon 34 are interacting. One or more sensors may be provided on or in the balloon in addition to the imaging apparatus 40 to characterise the interaction of the balloon 34 and vessel 10, particularly in relation to how the balloon 34 is inflating. Given that the occlusion 12 and vessel 10 may apply different forces at different circumferential and longitudinal points on the balloon 34, being able to understand the balloon's inflation beyond what can be gathered from the imaging apparatus 38 is highly beneficial.

In particular, it is important to ascertain that the balloon 34 has truly reached the target aspect ratio, that the balloon 34 is not kinked or in some way under-inflated, and/or where the greatest force is being exerted by the balloon 34. In addition, determining the configuration within the central region of the balloon 34, as well as along its length where possible, can be useful as these interactions may differ depending upon the relative location of the balloon 34 and the occlusion 12.

One or more of several different sensing mechanisms for characterising the interaction between the balloon 34 and the occlusion 12 may be used.

FIGS. 6 to 8 show embodiments of the balloon 34 that include arrangements of sensors on the internal or external surface of the balloon 34. This is in contrast to the embodiments of FIGS. 3 to 5, where the balloon 34 is shown without sensors. As will be appreciated, the provision of a non-compliant balloon 34 whose internal pressure is correlatable with a force applied to a lumen, and the methodologies surrounding the use and testing of the balloon is a core concept of the present application. The addition of sensors improves the certainty of the measurements for the medical practitioner.

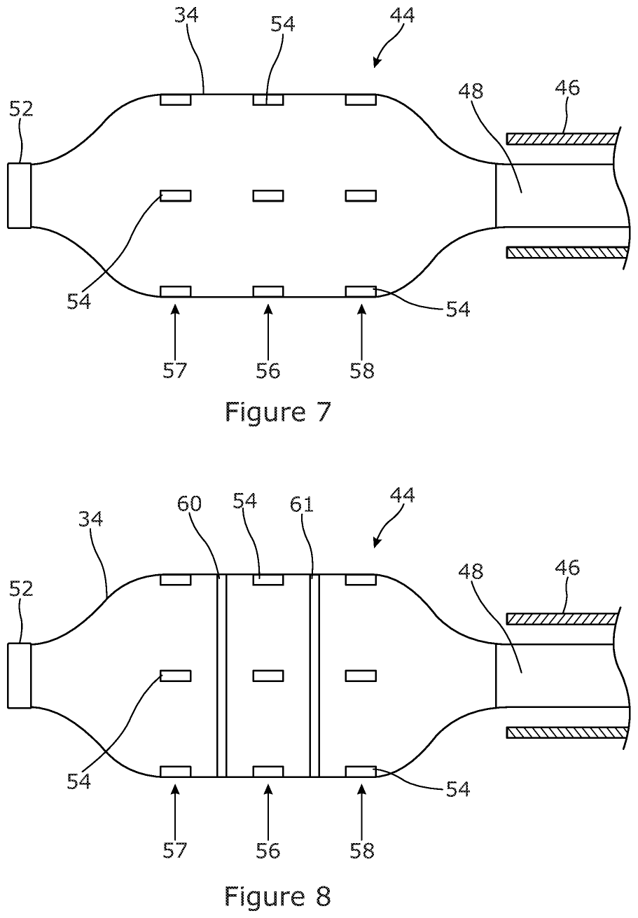

FIGS. 6 and 7 illustrate two embodiments incorporating contact sensors 54 onto the balloon 34. In FIG. 6, a band 56 of contact sensors 54 is provided around the circumference of the balloon 34 at its centre. The contact sensors 54 are evenly spaced around the circumference of the balloon 34. In FIG. 7, three bands 56, 57, 58 of contact sensors 54 are provided around the circumference of the balloon 34. The bands 56-58 of contact sensors 54 are spaced evenly longitudinally along the balloon 34. It will be appreciated that two bands, or more than three bands of sensors may be provided as desired. In some embodiments, the sensors 54 may not be arranged in bands but may be positioned in other ways around the balloon. Similarly, although in FIG. 7 the bands 56-58 of sensors 54 are longitudinally aligned, in other embodiments the bands of sensors may be staggered relative to one another.

These contact sensors 54 may be configured to detect electrical impedance or resistance, therefore allowing determination of when the balloon 34 is and is not in contact with the wall of the vessel 10. When the balloon 34 is in contact with the vessel at all points on its circumference, the balloon 34 and vessel 10 should have reached an aspect ratio of substantially 1.0. The practitioner may use the impedance sensors to understand the orientation of the balloon 34 within the vessel 10 and to determine where there is not contact being made and why. Each contact sensor 54 typically comprises an electrode supplied with a direct current and configured to measure the resistance through the electrode. The resistance of the electrode changes with changes in contact between the electrode and a surface.

By incorporating more sensors 54 around a particular circumference, the positions at which the balloon 34 is not in contact with the vessel 10 can be more accurately determined. The arrangement of FIG. 7, with longitudinally-spaced bands 56-58 of sensors 54, allows the surface contact with the occluded region of the vessel 10 to be monitored as well as the surface contact with the regions either side of the occluded region. This is because it is expected that the occluded region will be contacted by the central circumference of the balloon 34, and that the balloon 34 will extend either side of the occluded region.

Based on the change of impedance and/or resistance within the electrodes, the pressure applied between the vessel 10 and the balloon 34 may also be determined. Therefore, the contact sensors 54 may be used as both contact sensors and pressure sensors to give another means for determining the force required by a stent 20. In some embodiments, the balloon tolerances may be less strict if pressure values are also measured using sensors such as these. In some embodiments, separate force or pressure sensors may be incorporated into the balloon to characterise the force between the balloon and vessel.

FIG. 8 illustrates the bands 56-58 of contact sensors 54 with two additional profile sensors 60, 61 positioned between the bands 56-58. One profile sensor 60 is provided between the left-hand and centre contact sensor bands 57, 56 and the other profile sensor 61 is provided between the centre and right-hand contact sensor bands 56, 58. The profile sensors 60, 61 extend around the circumference of the balloon 34. Although the profile sensors 60, 61 are shown here in conjunction with the contact sensors, it will be appreciated that they could, in other embodiments be used in isolation or with different types of sensors. Similarly, although they are here shown to be positioned between the bands of contact sensors, they may, in other embodiments be positioned elsewhere. In other embodiments different numbers of profile sensors may be provided. In some embodiments, no profile sensors are provided. In others, one profile sensor is provided. In yet further embodiments, a plurality of profile sensors are provided.

The profile sensors 60, 61 are provided to enable determination of the profile of the balloon 34 during inflation. As before, profile here is used to describe aspect ratio and diameter of the balloon 34, or more simply, size and shape. By determining size/diameter of the balloon 34, it can be determined when the balloon is fully inflated to its correct size. The profile sensors permit determination of the aspect ratio to ensure that the balloon inflates correctly around its circumference. Using imaging techniques alone, it may be difficult to see if the balloon is inflating incorrectly, for example if there are kinks in the balloon or if the balloon is caught up in the vessel. Profile sensors may comprise strain gauges

The above sensors may comprise one or more printed electrodes. A printed electrode sensor would typically be a printed strip of conductive material on a surface, typically an internal surface of the balloon. The sensor may be circumferential around the balloon.

When used for a profile sensor, the electrode may act as a strain gauge, and may comprise two separated halves with interspaced branches so that the capacitance between the two halves can be measured and the distance therebetween determined. The electrode may be circumferentially arranged around a section of the balloon, and, where an array of sensors is provided, the sensors may be spaced along the length of the balloon at regular intervals. The shape of the balloon along its length may be determined using a sensor array.

FIGS. 10 to 12 demonstrate how the balloon 34 of FIGS. 6 to 8 may be positioned in the retracted state within the introducing sheath 46 prior to deployment and inflation.

To complement the sensors, the capabilities of the imaging system 40 may be enhanced. The catheter body 44 may further incorporate one or more means for positioning the catheter shaft 48 and balloon 34 using the imaging system 40.

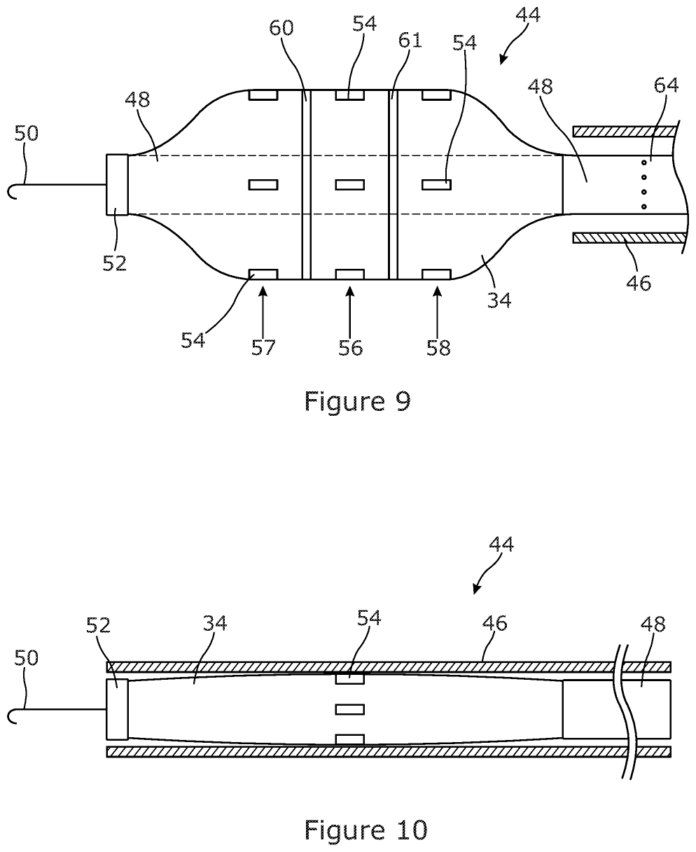

FIG. 9 shows the catheter body 44 of FIG. 8 being passed over a guide wire 50. The catheter body 44 of the embodiment shown in FIG. 9 also comprises a plurality of apertures 64 in the catheter shaft 48 for injecting a contrast agent or other fluid or visualization agent such as CO₂. As can also be seen in FIG. 9, indicated by the dotted lines, the catheter shaft 48 also passes through the balloon 34.

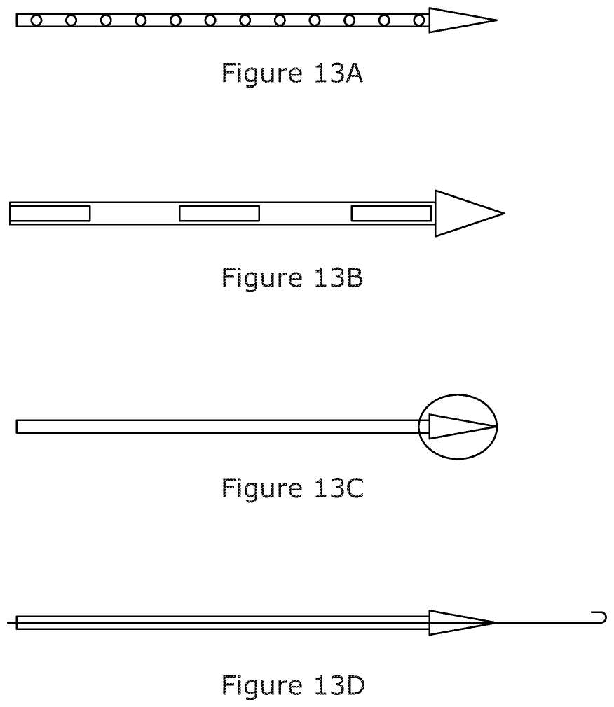

Positioning mechanisms may be provided on the sheath 46 or the catheter shaft 44 for use in cooperation with the imaging system. FIGS. 13A to 13D provide various different examples of these positioning mechanisms for aligning the balloon 34. As illustrated in FIG. 13A, the catheter may have radiopaque distance markers along its length for correct alignment. FIG. 13B illustrates how the catheter may also comprise ultrasound windows to permit IVUS visualization. Alternatives presented in FIGS. 13C and 13D are respectively that the nose of the catheter may be radiopaque and flexible and that the catheter may be advanced over a guide wire for correct positioning. In other embodiments, the attachment points of the device may each have a radiopaque marker to provide an indication of the locations of the attachment points relative to one another.

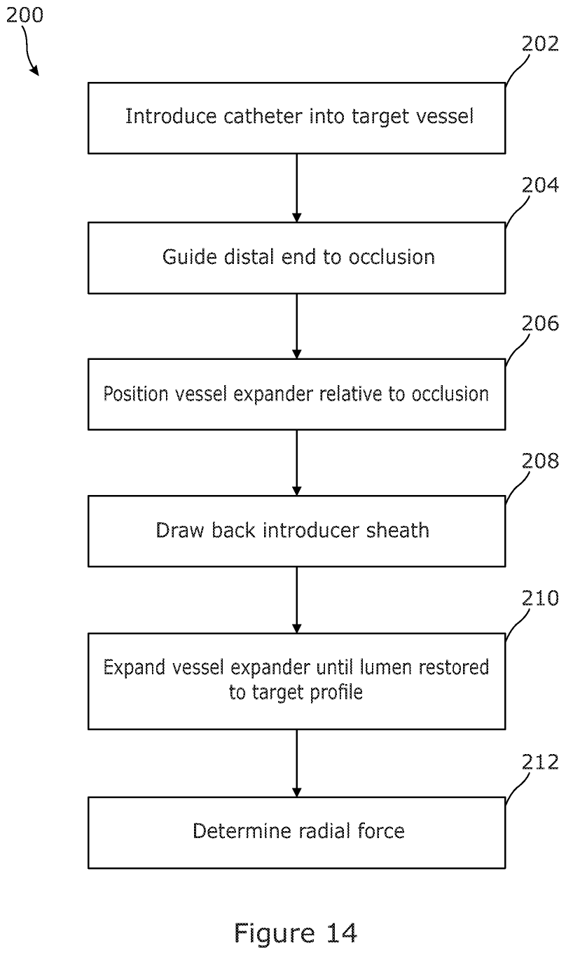

Although discussed in tandem with the device above, the methods of deploying and use of the device will now be discussed. In general, the device may be deployed and utilized for determining radial force by the steps shown in FIG. 14. While the expandable member in the method discussed below is a balloon, it will be appreciated that the method may also be performed using another type of expandable member instead of a balloon.

Before the method 200 of FIG. 14 is begun, it is assumed that a target vessel with a compression has been identified. The identification of the vessel is performed using the imaging system, and may include a venogram using magnetic resonance of computerized tomography techniques. Prior to the insertion of the catheter, other preparatory steps may also be performed. For example, other balloon-based catheters may be used to break up any stenosis present in the target vessel or to otherwise prepare the vessel. In other examples, guide wires may be passed through the occlusion to guide the catheter of the device. Of course, while not mentioned here, all preparatory steps to prepare the patient for receiving the catheter are also performed.

In addition, any preparatory measurements are also taken prior to the introduction of the device. Preparatory measurements, which are discussed in more detail in relation to later methods, may include determining an aspect ratio and diameter of the target vessel elsewhere other than the occlusion, i.e. its normal luminal dimensions. Based on these determinations, an appropriate balloon can be selected for use in the method.

Selecting an appropriate balloon may be performed by looking at imaging from an IVUS or other venographic imagery. From these images, an initial assessment of the vessel diameter may be determined for a normal sizing, an abnormal sizing, and an adequate, desired balloon sizing. Based on these sizings, an appropriate balloon can be selected from a range of balloons having distinct sizings and based on normal vessel sizes for the patient's medical information. The normal vessel size may be based on the patient age, weight, sex, and/or other characteristics. The normal vessel size may also be determined specifically for the patient by measuring the size of the vessel where there is no dilation due to congestion. Based on the normal vessel sizing and the available balloons, a balloon capable when dilated of achieving an aspect ratio of 1 having the vessel size of the healthy part of the vessel is chosen.

In the method 200 of FIG. 14, at step 202, the catheter body 44 of the device 32 according to the invention is introduced into the target vessel 10. The catheter body 44 is introduced into the target vessel 10 via an entry puncture site and any access vessels between the entry site and the target vessel. The handle 42 is maintained externally to the patient. At this stage, the balloon 34 is folded and deflated, and provided within the introducer sheath 46.

At step 204, the distal end of the catheter body 44 is guided to the target vessel 10 and the occlusion 12. The guiding of the catheter body 44 may be performed using the imaging system 40, and/or any of the positioning means discussed in relation to FIGS. 13A to 13D. As the balloon 34 is disposed at the distal end of the catheter body 44, guiding the distal end of the catheter body 44 brings the balloon 34 into proximity with the occlusion 12.

At step 206, the balloon 34 is positioned relative to the occlusion 12. The distal end of the catheter body 44 has already been guided close to or into the proximity of the occlusion 12, and now a fine-tuning of the positioning is performed. Based on visual data from the imaging system 40, the balloon 34 is positioned so that it is aligned with the occlusion 12 and so that its centre is centrally positioned relative to the occlusion 12. This is done so that the forces applied to balloon 34 when inflated are distributed as evenly as possible. Where sensors are provided in the balloon 34, centrally locating the balloon 34 ensures that the sensors are correctly positioned relative to the occlusion 12. The sensors may be marked using a positioning means such as those discussed in relation to FIGS. 13A to 13D, which may also be used for fine-tuning of the balloon's positioning relative to the occlusion.

At step 208 the balloon 34 is deployed from the sheath ready for inflation by withdrawal of the introducer sheath 46.

The balloon 34 is now in position to allow for determination of radial force. At step 210, the balloon 34 is inflated. The balloon 34 is inflated until the correct size and shape of the lumen of the target vessel 10 is restored to normal shape and size as identified prior to inserting the balloon 34. As noted above, the correct size and shape may be determined based on imagery from the imaging system 40 and/or based on readings from sensors provided on the balloon 34.

Once the desired shape and size of the balloon 34 is reached, the radial force experienced by the balloon 34 at that shape and size is determined at step 212.

The inflation of the balloon 34 may be performed in several ways. The balloon 34 may be inflated by incrementally increasing the pressure within the balloon 34 to set points. The set points may be predetermined set points or set points determined during the procedure by the user of the system. At each set point, the pressure is known, and it can be determined whether the size and shape of the lumen is restored. This determination may be made based on evidence of the imaging systems or an IVUS within the catheter, or based on one or more output signals from sensors.

Where sensors are provided in the balloon, step 210 may comprise increasing pressure to a set point, recording the pressure or output of the sensor(s), determining the shape and size of the balloon at that pressure based on the pressure or output of the sensor(s), and comparing the shape and size with the normal shape and size of the lumen. If the shape and size based on the sensor reading matches the shape and size of the lumen without an obstruction, the balloon is at the desired size.

Following a first inflation of the balloon, the balloon may be deflated and reinflated. Multiple inflations may be useful to determine the residual compression on a vessel separate from the initial dilation, for example to dilate and stretch a fibrotic lesion. Multiple inflations may be provided at a single position. The catheter may be moved a short distance and inflated again to gain another measurement of radial expansion force at a different position relative to the occlusion. Based on measurements gained along the length of an occlusion using the catheter at different points, an appropriate single value may be determined that characterises the radial expansion force required to suitably displace the occlusion along its length.

Having determined a radial force, a method for selecting a stent may be performed. Stents may be characterised by their ‘chronic outward force’, i.e. the amount of radial force they exert outwardly on the vessel, or by their ‘radial resistive force’ i.e. the amount of radial force they are configured to withstand from the vessel. Accordingly, the method of selecting a stent comprises determining a radial force required for a stent in the target vessel, obtaining a radial force of one or more stents, and choosing from the one or more stents the stent having the most appropriate radial force. The stent selected may be a primary stent, for initial placement within the target vessel, based on manufacturer-provided data relating to radial expansion force, or may be a secondary stent, comprising a stent element configured to reinforce a primary stent.

In order to determine the radial force exerted by a stent, each stent will have been characterised. The crush resistance and local resistance of the stent may have been tested and characterised using the methods described in ‘Endovascular Treatment for Venous Diseases: Where are the Venous Stents?’ A. Schwein et al, Methodist DeBakey Cardiovascular Journal 14 (3) 2018.

While the method above is described in relation to a vessel with an obstruction only, the method may also be performed within an existing stent to either test its usefulness, or if the existing stent is somewhat collapsed, to determine the radial force required for a secondary stent or a stent element for placement within the existing stent.

Similarly, while the balloon is here used alone, if a flexible primary stent is to be provided in the vessel that will be subsequently reinforced with stent elements or a secondary stent that reinforces the primary stent, then the balloon may serve the dual purpose of determining the radial force required for the secondary stent to reinforce the primary stent and of positioning and deploying the primary stent within the vessel. As the balloon expands to the diameter that the reinforcing stent elements will eventually have, a dual purpose of deploying the primary stent and measuring the requirements for the stent elements is useful in ensuring that the primary stent also has the correct diameter when deployed.

In some embodiments, the expandable member comprises basket catheter. Examples of basket catheter expandable members are shown in FIGS. 15 to 17. FIGS. 15 to 17 each schematically show the distal end of the catheter and the proximal end of the catheter below the distal end schematic. The proximal end of each comprises part of the central shaft and the handle.

As shown in FIG. 15, the catheter 132 is substantially similar to the catheter 32 including the expandable balloon 34. Catheter 132 has a rounded, atraumatic tip 53, is passed over a guide wire 50 and comprises an introducer sheath 46 and a central shaft 48. Where the catheter 32 of FIGS. 3 to 12 has a balloon 34 and inflation lumen (not shown) extending along the shaft 48, the catheter 132 of FIGS. 15 to 17 instead comprises an expandable basket 134 between the tip 53 and the shaft 48. The basket 134 is comprised of a plurality of flexible splines 135 extending longitudinally between the shaft 48 and the tip 53 and arranged radially about the central axis of the shaft 48.

A rod 137 extends coaxially through the central shaft 48 from the handle 142 and is fixed to the tip 53. The rod 137 is movable relative to the central shaft 48 in a slidable manner. The rod is provided within a protective shaft 139 indicated here using dotted lines. Retracting the rod 137 moves the tip 53 closer to the shaft 48, bending the splines 135 of the basket 134. The splines 135 flex outwardly as shown in FIG. 15. In bending, the splines 135 apply a force to the vessel 10. The force required to move the target vessel 10 to the target profile using the basket 134 can be determined based on the force applied to the tip 53 to achieve the bending of the basket splines 135. As can also be seen in FIG. 15, in the schematic of the handle 142, the retractable rod 137 extends through the catheter shaft 48 to the handle 142, where it can be controlled using a thumb button 143. The thumb button 143 is configured for reciprocal translation under manual control along the handle 142 to move the rod 137 back and forth and in so doing move the tip 53 back and forth longitudinally relative to the catheter shaft 48. A force determination can be made via a sensor (not shown) connected to the proximal terminus of the rod 137. In the embodiment shown in FIG. 15, a sensor is located in the handle that is adjoined to a spring 145 located at the proximal terminus of the rod 137. The sensor may be a strain gauge, such as an electrical strain gauge or a newton meter, or another type of force sensor. The force sensor may be connected to a processor.

The indication of force applied to the rod to displace the occlusion may be reflected on the handle as a spring force gauge; displacement of the spring being proportional to the force applied to the basket and vessel wall. A basket catheter is useful as it may be able to achieve a large range of diameters. The basket configuration also allows imaging like intra vascular ultrasound (IVUS) to be used during basket deployment as well as allowing the flow of blood in the vessel. A spring force gauge or other indicators of force may be used in other embodiments based on data output from sensors such as the pressure measurement in the balloon-based catheter.

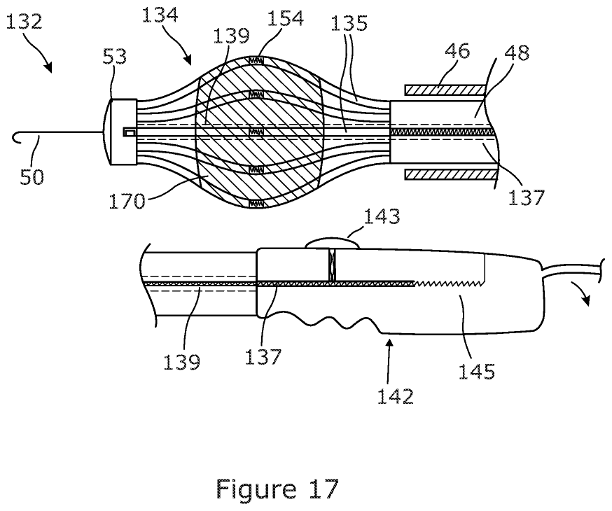

As can be seen in an embodiment depicted in FIG. 16, contact or pressure sensors 154 may also be incorporated into this design on each spline 135. In FIG. 17, a covering 170 is provided around the splines 135 to evenly distribute the force applied by them.

In any of the above catheters, an injection port connected to the outer sheath or a further hypotube or catheter shaft may be provided as part of the catheter through which a contrast medium can be injected to permit visualisation of the vein while expanding the expandable member. It will also be appreciated that the marking systems of FIG. 13 may also be applied to a catheter comprising a basket.

In one or more embodiments, force-mapping software may be provided to permit a medical practitioner using a catheter device as described herein to accurately track force measurements within a patient's anatomy. Using the software, the practitioner may select a location at which the catheter device has been used to measure a force overcoming an occlusion and to enter data relating to the measurement performed. As the catheter device is advanced or withdrawn through the target vessel, further measurements may be performed and registered in the software. The software may be configured to receive data output from the catheter device to permit registration of the correct data at the correct location. In relation to location, the software may create a model of the patient from imaging data created prior to the use of the catheter device, or may update a generic model based on measurements and inputs from the practitioner or directly from the catheter device. The software may be configured to permit identification of the beginning of structures within the patient such as the access point, the ends of the catheter, and structures such as start and end points and paths of vessels including the internal iliac vein, the external iliac vein, the common femoral vein. Points of flexion of the patient may also be indicated. An IVUS system may be utilised for this locating, as is discussed further below.

The software may be configured to receive data relating to the target vessel such as diameter of the target vessel along its length, dimensions of the occlusion, dimensions of the wall of the vessel such as thickness. These dimensions may be calculated based on data from the imaging systems and using image processing techniques. Dimensions such as the occlusion dimensions and diameter of the vessel may be determined based on the point of first contact between the expandable member and the target vessel. Where contact sensors are utilised, the first contact between expandable member and vessel may be registered using a signal from the contact sensors. Once a signal from the contact sensors is identified, the diameter of the expandable member can be determined, with the relevant dimensions determined based on the size of the expandable member. Before the first contact, the relative position or diameter of the expandable member can be determined based on the pressure (for a balloon) or force (for a basket) at that moment. Where contact sensors are not used, the first contact may be determined based on the force or pressure measurements, based on signals from profile sensors, or based on imaging data. For example, the expansion of the expandable member may be smooth until the first contact is made, at which point the rate of expansion may change, and this may be determined based on the change in pressure or force over time.

The software may associate locations with images of that location within the body. To aid the determination of a stent, the software may determine a force to overcome an occlusion based on the measurements input to it. The software may compare the force measurement against known radial force values for a preselected set of stents and select the most appropriate stent to apply the radial force. The medical practitioner may also choose a stent based on the force.

The software may be provided to be run on a computer, or may be provided within standalone hardware in a plug-and-play arrangement comprising a processor, a display device, and input/output ports for data input and output. The catheter device may be connected directly to the plug-and-play box, along with the imaging system. There may also be provided in the box an output for sending video data to another display device. This system may be integrated with a fluoroscopy system so that fluoroscopy imaging data and IVUS imaging data may be aligned and overlaid based on fiducial points on the body.

In some embodiments, an IVUS system may be provided within the lumen of the introducer or through a central lumen in the catheter shaft. The IVUS system may be used to measure length of a target vessel or portion of the target vessel to inform stent length, or distance moved along a target vessel from an access position. This data may also be output to the software to determine a location at which a force is being applied relative to the access point. The IVUS system may also determine a length of the occlusion to which the force is applied.

In some embodiments, means other than the IVUS system may be utilised to determine length of or of a portion of the target vessel to identify how long the selected stent should be. These means may comprise one or more markers distinguishable from the catheter shaft in some way and movable along the shaft. The marker may be distinguished by colour, by a distinctive pattern, or otherwise. The marker may be moved up and down the shaft from the handle to mark how far a catheter is moved along a vessel. In some embodiments, other types of markers may be used—the shaft may have measurement points on its surface. Using these means, a length can be determined using the catheter device. Once the distal end of the catheter is disposed within the target vessel, the tip of the distal end can be positioned at a most distal point of the occlusion. The most distal point of the occlusion may be determined based on imagery from the imaging system and/or the IVUS. The expandable member is deployed and expanded enough to touch the walls of the vessel and occlusion. The expandable member and catheter as a whole is then pulled back through the vessel. The slightly-expanded expandable member tracks the contour of the vessel. Expanding the member in this way forces the centre of the shaft of the catheter to track the route along the vessel, thereby giving a more precise measurement of the length than would be achieved if the member were not expanded. Once the end point of the occlusion or whichever other position is to be the end of the stent within the vessel, the distance that the catheter has been pulled back is determined, and this is established as the desired stent length.

One or more pressure sensors may be incorporated onto the hypotube and/or central shaft and/or introducer shaft to determine pressure within the vessel. One or more pressure sensors may be incorporated into a tip of the catheter device and/or on the expandable member to determine pressure within the vessel. Determining pressure within the vessel is useful in comparing with the pressure or force applied to the expandable member, as well as in determining the dilation of the target vessel and therefore the effect of the occlusion on blood flow. The determination of characteristics of blood flow may be useful in determining the expected blood flow once patency is restored. For the basket catheter, these characteristics may also be useful in determining the point at which the target profile has been achieved. The pressure sensor may comprise a piezoelectric sensor, such as a MEMS pressure sensor, configured to measure the fluid pressure within the lumen of the vessel.

In embodiments comprising an expandable member in the form of a basket, the splines may be adapted to enable tracking of the internal profile of the vessel wall. A sensor may be incorporated to monitor movement, flexing, or distance from a longitudinal axis of the splines to determine the vessel profile. For example, the splines may be sprung or spring-mounted. When deployed in this spring mounted form, the splines expand to the diameter of the vessel and contact the vessel wall directly. The distal end of the device may be advanced in a vessel to a location beyond a partial occlusion or constriction of the vessel. The expandable member may be deployed and then withdrawn from the advanced position back through the partial occlusion while the splines are expanded such that the splines follow the contours of the vessel wall. By measuring the output of a sensor configured to measure this movement, the topography of the vessel wall can be determined.

Such a system may make use of one or more wires connected to the splines that move longitudinally relative to the shaft as the splines move. By measuring the movement the wires through the elongate body of the catheter, the change in the wall diameter can be tracked, and a radius or diameter determined for the vessel. Laser measurement systems may also be utilised to make this measurement within the handle at the proximal terminal region.

Therefore, from a combination of the force-measurement, the pressure-measurement, and the diameter-tracking systems described above, a series of outputs may be used to form computer models of the vessel. The output of the strain gauge or force sensor used in combination with the splines permits the radial expansive force at discrete points along the length of the vessel to be determined. The output of the pressure sensor permits the hydrostatic pressure of the fluid flow (e.g. blood pressure) to be identified at points along the vessel. The output of the expansion monitoring using the sprung splines enables the profile of the vessel to be recorded.

From each of these, a map may be determined. Accordingly, a force map, a pressure map, and a topography map may be generated in silico for a region of the vessel that is to be stented. An algorithm may utilise each of these maps as inputs for generating a computer model of the vessel in question. The computer models may be interrogated to inform stenting strategy for the patent. For example, a further stent selection algorithm may apply virtual stent models to the vessel model at different lengths and widths to determine the optimal stent to apply in the vessel. In addition, the map of the vessel may be correlated to landmarks within the anatomy of the patient, such as the main vessels, branch vessels, the pelvis, spine, inguinal ligament etc to enable choices to be made about which stent to choose.

To ensure repeatability, the catheter device may be connected to a motor configured to incrementally or continually move the catheter within the vessel. The motor may be configured to withdraw the catheter over a set interval distance or at a predetermined speed to allow accurate measurements to be made.

In general, the vessels in which the above methods and devices are used will be in the venous system, i.e. veins, although the techniques herein may be applied to other vessels. For use in veins, the expandable member may be limited in the maximum size it can achieve to restrict overexpansion of the vein which may cause damage in some cases.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims. In addition, the above described embodiments may be used in combination unless otherwise indicated.

1. A catheter-based device for determining the radial expansion force required to displace an occlusion in a vessel located in a subject, the device comprising:
an elongate body defining a proximal and a distal termini, the body comprising a sheath that encloses a hollow lumen within, which extends along substantially the full length of the body,
the proximal terminal region comprising:
a user-interfacing hub, the hub comprising a handle for maneuvering the body and configured for handling by an operator;
a control interface for controlling the device; and
a sensor configured to measure one or more parameters relevant to a force applied to the vessel by the device;
the distal terminal region comprising:
an expandable member movable between a retracted position, in which the expandable member is within the hollow lumen, and a deployed position, in which the expandable member is disposed beyond the distal terminus, and controllable via the control interface to expand radially;
wherein, the expansion of the expandable member is correlated to a defined radial expansion force value.

2. The catheter-based device of claim 1, wherein the expandable member comprises a non-compliant balloon, wherein the balloon is expandable by passing pressurised fluid along a lumen to the interior of the balloon, wherein the sensor comprises a pressure sensor configured to measure a pressure of the fluid in the balloon, and wherein the pressure is correlated to a defined radial expansion force value.

3. The catheter-based device of claim 1, wherein the expandable member comprises a basket having a plurality of longitudinal splines, wherein the basket is expandable by bringing the ends of the basket towards one another, wherein the sensor comprises a force sensor configured to measure a force applied to the basket to bring its ends together, and wherein the measured force is correlated to a defined radial expansion force value.

4. The catheter-based device of claim 3, wherein the expandable member comprises a fabric covering at least partially surrounding the basket.

5. The catheter-based device of claim 3, wherein the plurality of longitudinal splines extend between a shaft forming part of the elongate body and a distal tip, the device further comprising a rod extending along the elongate body that is movable relative to the shaft and fixedly connected to the distal tip for applying a force to the distal tip to bring the ends of the basket together, and wherein the force sensor is configured to measure the force applied to rod.

6. The catheter-based device of claim 5, wherein the control interface comprises a thumb button connected to the rod for reciprocal translation of the rod.

7. The catheter-based device of claim 1, the distal terminal region further comprising one or more occlusion sensors configured to characterise occlusion.

8. The catheter-based device of claim 7, wherein the one or more occlusions sensors are provided on a surface of the expandable member.

9. The catheter-based device of claim 7, wherein the one or more occlusions sensors comprise contact sensors.

10. The catheter-based device of claim 7, wherein the one or more occlusions sensors comprise pressure sensors.

11. The catheter-based device of claim 7, wherein the one or more occlusions sensors comprise profile sensors.

12. The catheter-based device of claim 7, wherein the one or more occlusions sensors are positioned at the longitudinal centre of the expandable member.

13. The catheter-based device of claim 1, further comprising an imaging system.

14. The catheter-based device of claim 1, wherein the catheter-based device is configured to be passed over guide wire.

15. A method for determining the radial expansion force required to displace an occlusion in a vessel located in a subject, the method comprising:
providing a catheter-based device having an expandable member expandable to apply force to the occlusion;
disposing the expandable member within the vessel in the region of the occlusion;
expanding the expandable member to achieve a target profile within the lumen, wherein the expansion of the expandable member is correlated to a defined radial expansion force value; and
determining the radial expansion force value applied by the expandable member to the lumen to achieve the target profile based on the correlation.

16. The method of claim 15, wherein the vessel is a vein.

17. The method of claim 15, wherein the expansion of the expandable member is limited based on the target profile.
